(19) United States
(12) Patent Application Publication
(10) Pub. No.: US 2020/0077507 A1
(43) Pub. Date: Mar. 5, 2020

(54) DC Constant-Field Synchrotron Providing Inverse Reflection of Charged Particles

(71) Applicant: Massachusetts Institute of Technology, Cambridge, MA (US)

(72) Inventor: Alexey L. Radovinsky, Cambridge, MA (US)

(73) Assignee: Massachusetts Institute of Technology, Cambridge, MA (US)

(21) Appl. No.: 16/500,303

(22) PCT Filed: Apr. 20, 2018
(86) PCT No.: PCT/US2018/028596

§ 371 Date: Oct. 2, 2019

Related U.S. Application Data
(60) Provisional application No. 62/488,153, filed on Apr. 21, 2017.

(51) Int. Cl. H05H 13/10; H05H 13/04; H05H 15/00; H01F 6/06

(52) U.S. Cl. CPC H05H 13/10; H05H 13/04; A61N 2005/1087; H01F 6/06; H05H 15/00

(57) ABSTRACT

Charged particles are accelerated in a direct-current synchrotron, wherein a plurality of achromatic magnets define an acceleration device. A beam of charged particles is directed toward one of the magnets, and the charged-particle beam penetrates a gap in the magnet and is repeatedly redirected through an arc of at least 270° via inverse reflection at each of the achromatic magnets to produce a series of beam lines that form a circuit in which the charge-particle beam is accelerated over successive passes through the circuit. The achromatic magnets generate a constant magnetic field. The charged particles can then be extracted from the acceleration device.

### GOVERNMENT SUPPORT

This invention was made with government support under Grant No. R44 CA203450 awarded by the National Institutes of Health and under Grant No. OE-SC001 3499 awarded by the US Department of Energy. The US Government has certain rights in the invention.

### BACKGROUND

Superconducting ion beam accelerators are increasingly used for hadron radiotherapy treatment (HRT). Thus far, the most important requirements to the accelerators employed in the HRT systems were compactness, low cost and ease of operation. Cyclotrons and to some extent synchrotrons are the best fit for these requirements.

Synchrotrons and, particularly, cyclotrons, however, are usually designed for accelerating only one type of charged particle, whereas medical indications for different types of cancer tumors often require using different ionized particles. Unique designs permitting acceleration of different types of particles are very complex and need an elaborate retuning for transition between the different particles. This limitation precludes these systems from being used in general hospital practice and limits their field of application to high-tech physical laboratories and affiliated medical centers. Additionally, the choice of charges particles and their extraction energy is uniquely limited by the design of these types of accelerators.

Another desired feature of HRT accelerators is beam extraction at variable energies permitting in-depth scanning of the tumor without secondary radiation caused by currently used for changing the beam energy degraders. This feature is easily implemented in synchrotrons, and recent inventions [see L. Bromberg, J. V. Minervini, P. Le, A. Radovinsky, P. Michael, T. Antaya, “Ultra-Light, Magnetically Shielded, High-Current, Compact Cyclotron,” U.S. Pat. No. 8,975,836 B2 (Mar. 10, 2015) and A. L. Radovinsky, et al., “Variable Energy Acceleration in a Single Iron-Free Synchrocyclotron,” PSFC MIT Report, PSFC/RR-13-9 (Sep. 5, 2013)] of a variable energy iron-free cyclotron made beam extraction at variable energies possible in cyclotrons, as well.

Existing designs of both synchrotrons and variable energy iron-free cyclotrons, however, require changing the magnetic field for transition from one beam energy to another between the shots in the case of cyclotrons and during the very short time of flight (ToF) in synchrotrons. The latter practically excludes the possibility of using superconducting magnets in existing designs of synchrotrons. This limits the field in these synchrotron magnets to the maximum achievable using normal magnets and precludes reducing the size of the system by going to higher fields typical for the superconducting magnets.

### SUMMARY

A DC synchrotron and methods for its operation to accelerate charged particles are described herein, where various embodiments of the apparatus and methods may include some or all of the elements, features and steps described below.

Charged particles are accelerated in a direct-current synchrotron, wherein a plurality of achromatic magnets define an acceleration device. A beam of charged particles is directed toward one of the magnets, and the charged-particle beam penetrates a gap in the magnet and is repeatedly redirected through an arc of at least 270° via inverse reflection at each of four magnets to produce a series of beam lines that form a circuit in which the charge-particle beam is accelerated over successive passes through the circuit. The magnets generate a magnetic field that is substantially constant over time (constant-in-time)—e.g., deviating in time by no more than 1%—over the repeated redirections of the charged-particle beam. The charged particles can then be extracted from the acceleration device.

Embodiments of the synchrotron, described herein, can be free of the limitations inherent to the synchrotrons known so far. Features of the accelerator, described herein, can include any or all of the following: (a) acceleration of the particles from injection to extraction at a constant field from the magnets, which makes it possible to use superconducting magnets and to subsequently reduce the size of the system; (b) acceleration of various ion particles in the same device without any retuning of the magnets; (c) choice of the ions and the energy to which they can be accelerated can be limited only by the size and the field of the magnets; (d) using the same frequency-versus-time tuning of the RF drive for all accelerated ions with the only adjustment to be made being the voltage in the acceleration cavity of the RF drive; and (e) feasibility of fast-beam energy variation for the in-depth scanning due to not needing to vary the field in the magnets and it potentially being limited only by electronics.

These features can make the accelerator described herein a unique choice for use in HRT treatment centers. The accelerator can be made smaller than comparable-performance synchrotrons and, at the same time, can be easier to operate and far more versatile with respect to the selection of accelerated ions.

In another method, a path for charged particles is bent by first directing a charged-particle beam along a first beam line toward a first magnet generating a magnetic field. The path of the charged-particle beam is then redirected to produce a second beam line with the magnet via inverse reflection.

The path for the charged particles can then be further bent with a second magnet to produce a third beam line via inverse reflection. In an accelerator, the path can then be bent again by a third magnet to form a fourth beam line and by a fourth magnet to form a fourth beam line. This sequence continues over multiple revolutions from injection of the charged particles to their extraction. The first and the second magnet form the first achromatic pair. The third and the fourth magnets form the second achronatic pair. All beam lines between the fourth and the first magnets are collinear as well as those between the second and the third magnets. Beam lines between the second and the third magnet and between the third and the fourth magnets are parallel to each other. Different ions with different energies can be likewise reflected via inverse reflection at each of the magnets to produce the desired beam lines.

### BRIEF DESCRIPTION OF THE DRAWINGS

FIG. 1 shows the toroidal magnets of an exemplary DC synchrotron with coordinate axes.

FIG. 2 shows the layout of components in the embodiment of the DC synchrotron of FIG. 1.

FIG. 3 shows the trajectories of protons with inverse reflection at the toroidal magnets; the trajectories, which can be seen with progressively large penetrations into the toroidal magnets (increasing with increasing energy), are for 50 keV, 250 MeV, 750 MeV, and 1.25 GeV.

FIG. 4 shows the trajectories of carbon ions with inverse reflection at the toroidal magnets; the trajectories, which can be seen with progressively large penetrations into the toroidal magnets (increasing with increasing energy), are for 100 keV, 100 MeV, 200 MeV, 300 MeV, and 400 MeV.

FIG. 5 is an isometric view of a toroidal magnet.

FIG. 6 is an end view of a toroidal magnet.

FIG. 7 is a histogram of the magnetic field in the beam plane, XOZ.

FIG. 8 plots the B-filed along the radial X-coordinate, Y=Z=0.

FIG. 9 shows magnetic-field contours from a toroidal magnet from 10 gauss to 100 gauss via 10-gauss steps from a view along the Zaxis.

FIG. 10 shows magnetic-field contours from a toroidal magnet from 10 gauss to 100 gauss via 10-gauss steps from a view along the Yaxis.

FIG. 11 shows a trajectory of a 10-MeV proton, wherein the proton trajectory is redirected with 1-m-long magnets via direct reflection.

FIG. 12 shows a trajectory of a 10-MeV proton, wherein the proton trajectory is redirected with 1-m-long magnets via inverse reflection.

FIG. 13 shows a trajectory of a 10-MeV proton, wherein the proton trajectory is redirected with 2-m-long magnets via direct reflection.

FIG. 14 shows an isometric view of a toroidal magnet with iron shimming.

FIG. 15 shows an end view of the toroidal magnet of FIG. 14 with the iron shimming.

FIG. 16 shows a constant-field toroid (CFT) sub-assembly of enclosed racetracks of the magnet.

FIG. 17 shows a perspective view of a CFT magnet.

FIG. 18 shows a histogram of the filed in the beam space.

FIG. 19 plots the magnetic field of the magnet of FIG. 18 along the radial, X-coordinate, where Y=Z=0.

FIG. 20 shows the field contours about the CFT magnet at Z=0 from 10 gauss to 100 gauss via 10-gauss steps.

FIG. 21 shows the field contours about the CFT magnet at Y=0 from 10 gauss to 100 gauss via 10-gauss steps.

FIG. 22 is a schematic illustration of the pathway of an ion undergoing direct reflection.

FIG. 23 is a schematic illustration of the pathway of an ion undergoing inverse reflection.

FIGS. 24 and 25 show the trajectories of a proton at 1.25 GeV and a carbon ion at 400 MeV, wherein the image of FIG. 25 is a close-up of the upper left corner of FIG. 24.

FIG. 26 shows the Y(X) trajectories for 70, 150, and 230 MeV protons in an ideal Enge magnet.

FIG. 27 shows the Z(X) trajectories for the 70, 150, and 230 MeV protons in an ideal Enge magnet.

FIG. 28 shows a physical implementation of a toroidal Enge magnet (TEM).

FIG. 29 plots the magnetic field profile of the TEM of FIG. 28 along with an analytical field profile.

FIG. 30 shows a physical implementation of a CFT magnet.

FIG. 31 shows a subcoil of a TEM.

FIG. 32 shows a subcoil of a CFT magnet.

FIG. 33 shows a TEM magnetic-field profile.

FIG. 34 shows a CFT magnetic-field profile.

FIG. 35 shows an in-plane, Z(X), projection of ion trajectories in a TEM.

FIG. 36 shows an in-plane, Z(X), projection of ion trajectories in a CFT magnet.

FIG. 37 shows an out-of-plane, Y(X), projection of ion trajectories in a TEM.

FIG. 38 shows an out-of-plane, Y(X), projection of ion trajectories in a CFT magnet.

FIG. 39 is a plot of frequency versus time for a proton accelerated to 250 MeV in two 50-keV cavities.

FIG. 40 is a plot of frequency versus time for a carbon ion accelerated to 400 MeV in two 50-keV cavities.

FIG. 41 is an overlay of frequency-versus-time dependencies for a proton and a carbon atom.

In the accompanying drawings, like reference characters refer to the same or similar parts throughout the different views; and apostrophes are used to differentiate multiple instances of the same item or different embodiments of items sharing the same reference numeral. The drawings are not necessarily to scale; instead, an emphasis is placed upon illustrating particular principles in the exemplifications discussed below. For any drawings that include text (words, reference characters, and/or numbers), alternative versions of the drawings without the text are to be understood as being part of this disclosure; and formal replacement drawings without such text may be substituted therefor.

### DETAILED DESCRIPTION

The foregoing and other features and advantages of various aspects of the invention(s) will be apparent from the following, more-particular description of various concepts and specific embodiments within the broader bounds of the invention(s). Various aspects of the subject matter introduced above and discussed in greater detail below may be implemented in any of numerous ways, as the subject matter is not limited to any particular manner of implementation. Examples of specific implementations and applications are provided primarily for illustrative purposes.

Unless otherwise herein defined, used or characterized, terms that are used herein (including technical and scientific terms) are to be interpreted as having a meaning that is consistent with their accepted meaning in the context of the relevant art and are not to be interpreted in an idealized or overly formal sense unless expressly so defined herein. For example, if a particular composition is referenced, the composition may be substantially (though not perfectly) pure, as practical and imperfect realities may apply; e.g., the potential presence of at least trace impurities (e.g., at less than 1 or 2%) can be understood as being within the scope of the description. Likewise, if a particular shape is referenced, the shape is intended to include imperfect variations from ideal shapes, e.g., due to manufacturing tolerances. Percentages or concentrations expressed herein can be in terms of weight or volume. Processes, procedures and phenomena described below can occur at ambient pressure (e.g., about 50-120 kPa—for example, about 90-110 kPa) and temperature (e.g., −20 to 50° C.—for example, about 10-35° C.) unless otherwise specified.

Although the terms, first, second, third, etc., may be used herein to describe various elements, these elements are not to be limited by these terms. These terms are simply used to distinguish one element from another. Thus, a first element, discussed below, could be termed a second element without departing from the teachings of the exemplary embodiments.

Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly. The term, “about,” can mean within ±10% of the value recited. In addition, where a range of values is provided, each subrange and each individual value between the upper and lower ends of the range is contemplated and therefore disclosed.

Further still, in this disclosure, when an element is referred to as being “on,” “connected to,” “coupled to,” “in contact with,” etc., another element, it may be directly on, connected to, coupled to, or in contact with the other element or intervening elements may be present unless otherwise specified.

The terminology used herein is for the purpose of describing particular embodiments and is not intended to be limiting of exemplary embodiments. As used herein, singular forms, such as “a” and “an,” are intended to include the plural forms as well, unless the context indicates otherwise. Additionally, the terms, “includes,” “including,” “comprises” and “comprising,” specify the presence of the stated elements or steps but do not preclude the presence or addition of one or more other elements or steps.

Additionally, the various components identified herein can be provided in an assembled and finished form; or some or all of the components can be packaged together and marketed as a kit with instructions (e.g., in written, video or audio form) for assembly and/or modification by a customer to produce a finished product.

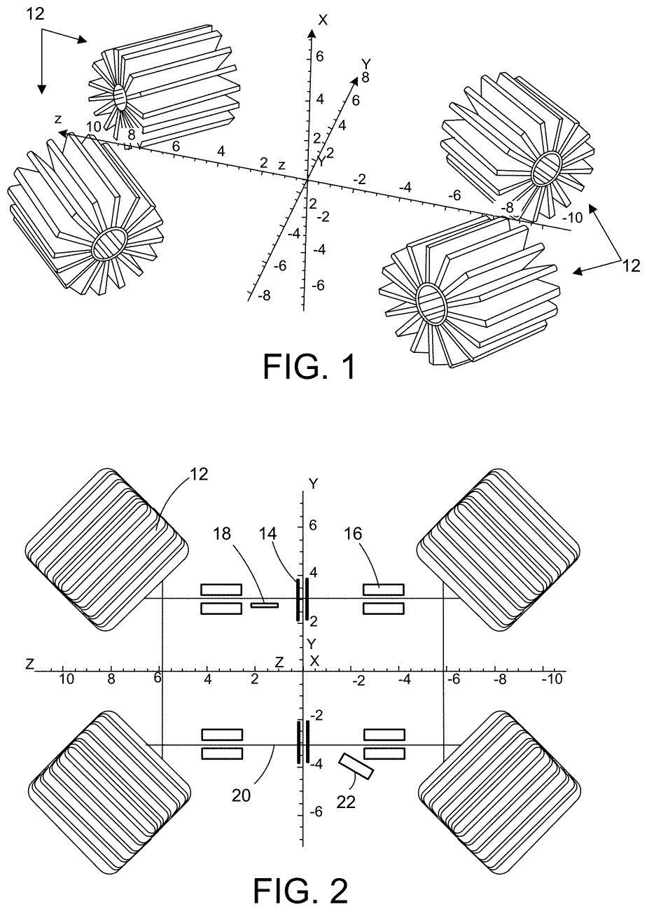

Embodiments of the accelerator can include four toroidal bending magnets 12 installed in a rectangular arrangement with axes in the same, ZOY, plane. The axes are tilted at 45 degrees, as shown in FIG. 1. FIG. 2 depicts the same system in more detail. It shows four magnets 12, two acceleration cavities 14, and focusing multipoles 16 installed around the beam line 20. Also shown are schematics of the ion source 22 and the extraction apparatus 18.

Toroidal magnets, two on the left and two on the right of FIG. 2, form achromatic pairs of magnetic mirrors, each reflecting the beam 20 by 180 degrees (i.e., to the opposite direction).

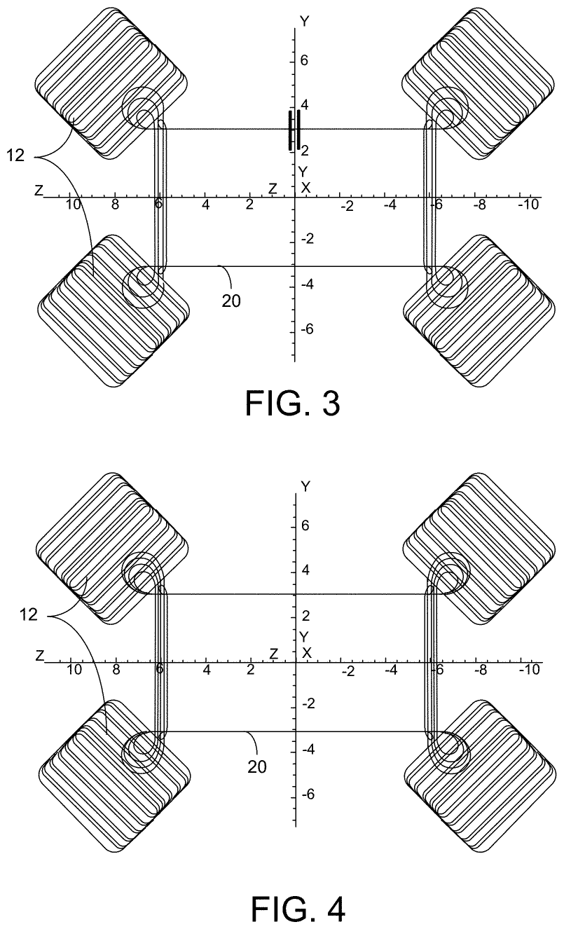

FIGS. 3 and 4 depict results of electromagnetic modeling of beam trajectories using the OPERA simulation software program [from Cobham PLC (UK)] for protons and carbon ions, ¹²C⁶⁺, for a wide ranges of energies, from 50 keV to 1.25 GeV for protons and from 100 keV to 400 MeV for carbon ions, respectively. The significance of the lower limit of modeled energies will be explained, below.

### Achromatic Toroidal Bending Magnets

The magnetic system comprises achromatic arrangements of the toroidal bending magnets (TBMs). Using toroidal field (TF) magnets for making bending achromats for the hadron therapy gantries was proposed recently by L. Bromberg and P. Michael, “Toroidal Bending Magnets for Hadron Therapy Gantries,” U.S. Pat. No. 9,711,254 B2 (Jul. 18, 2017).

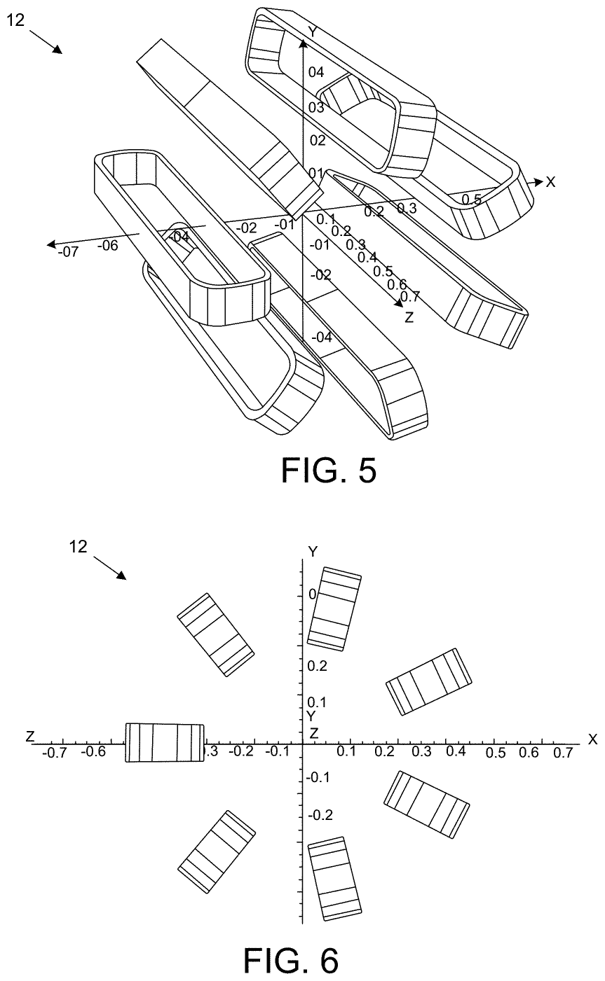

Each achromat includes two TF coils comprising racetrack windings, typically arranged as shown in FIGS. 5 and 6, and formed, e.g., of NbTi or another superconductor (e.g., a high-temperature superconductor, such as YBa₂Cu₃O₇ or Bi₂Sr₂Caₙ₋₁CuₙO₄₊₂ₙ₊ₓ), cooled (e.g., with a cryocooler or with liquefied helium) below its critical temperature (e.g., at 4.2 K for NbTi), are electrically connected with a power source. The superconductor can be in the form of 0.75-mm strands wound into a Rutherford cable (e.g., windings of 6 strands of NbTi and 6 strands of copper).

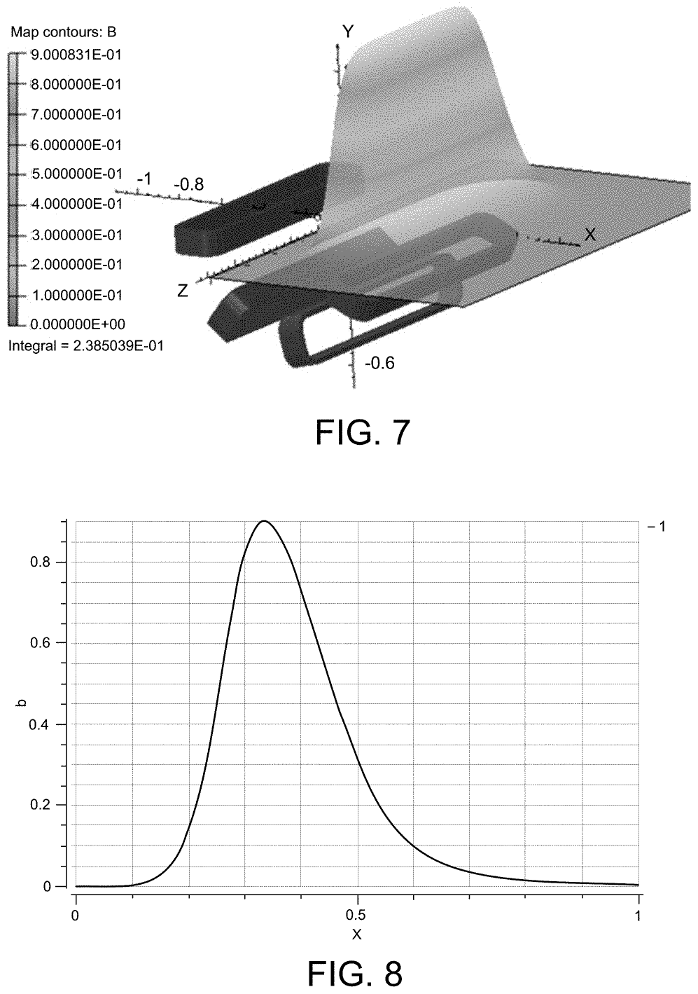

As shown in FIGS. 7 and 8, the field along the axial extent of the magnet is almost constant and is frequently considered to be two-dimensional (2D, varying only in radial and azimuthal, with respect to the magnet axis, directions). In the depicted example, the beam space is in the ZOX plane, equidistant from the symmetrical coils above and below this plane. Due to the symmetry, the field at the ZOX plane is unidirectional, normal to the plane and parallel to the Y-axis. The shape of the field along the radial, X-axis, at the mid-plane, XOY, is shown in FIG. 8.

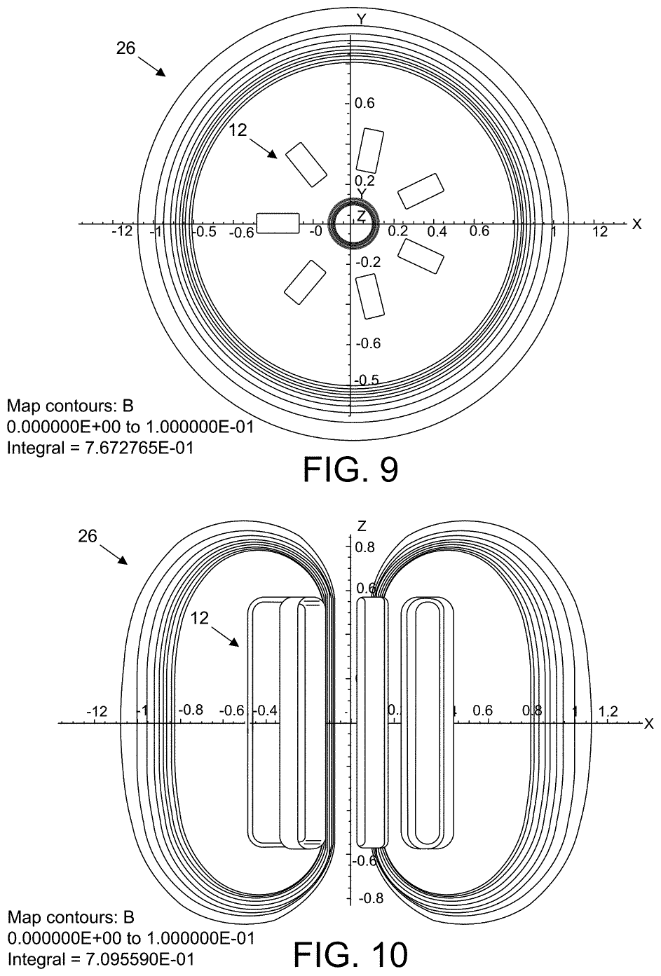

This type of toroidal field (TF) magnetic arrangement is characterized by very good field containment. FIG. 9 shows that the field of this 1-T magnet falls to the level of less than 10 gauss at about 1-m radial distance from its axis.

The quality of the beam optics of an achromatic magnetic mirror is strongly contingent on the uniformity of the 2D field distribution in the beam space along the axial coordinate. This feature provides for the equality of the incoming and outgoing angles of the particle trajectories, facilitating the high quality of the optics of the achromat. On the way through the magnet, the particle passes along the same path, symmetrical about the point of inflection, with the same magnetic-field-versus-distance variation on the way into and out of the magnet. For a single-energy particle, this equivalence can be achieved by positioning the magnet so that the point of inflection lies on the mid-plane, Z=0.

FIG. 10 shows that the constant-field contour lines 26 deviate from the straight (parallel to the magnet axis) line, slightly bowing outwards, symmetrically about the mid-plane (Z=0) of the magnet. This small deviation is harmful for the beam optics. At different energies, trajectories of the particles launched along the same incoming line have different coordinates of the inflection points (shifted along the Z-axis); and bowing of the field causes optical aberrations, resulting in non-parallelism of the beam lines outgoing from the achromatic magnet pair.

### Beam Focusing

Beam focusing can be accomplished by a traditional synchrotron method using multipole magnets installed along the sides of the system where the beam lines in the beam-path circuit are collinear at all ion beam energies. These are horizontal beam lines 20 in FIG. 2. The acceleration cavities are also positioned along these lines 20.

It shall be noted that the capability of these traditional methods of beam focusing are limited and need to be supplemented by the good focusing properties of the bending magnets, as described herein. The beam 20 comprises the ions moving with small deviations from the nominal, both energy-wise and direction-wise. This deviation causes a certain defocusing of the beam 20 at the exit of the bending magnet. Usually, defocusing is considered in two directions, horizontal (i.e., in the plane of the beam) and vertical (i.e., out of this plane).

The following discussion addresses focusing properties of the toroidal bending magnets (TBMs) 12, first with respect to their horizontal focusing properties and then with respect to their vertical focusing properties.

### Horizontal Focusing

In embodiments of the synchrotron, as described herein, the horizontal, in-plane, aberrations of the beam optics accumulate very fast, causing deviations of the trajectories from the ideal pattern and potentially leading to the eventual loss of the beam 20. Several possible methods of mitigating such optical aberrations are described below.

### Direct Versus Inverse Reflection

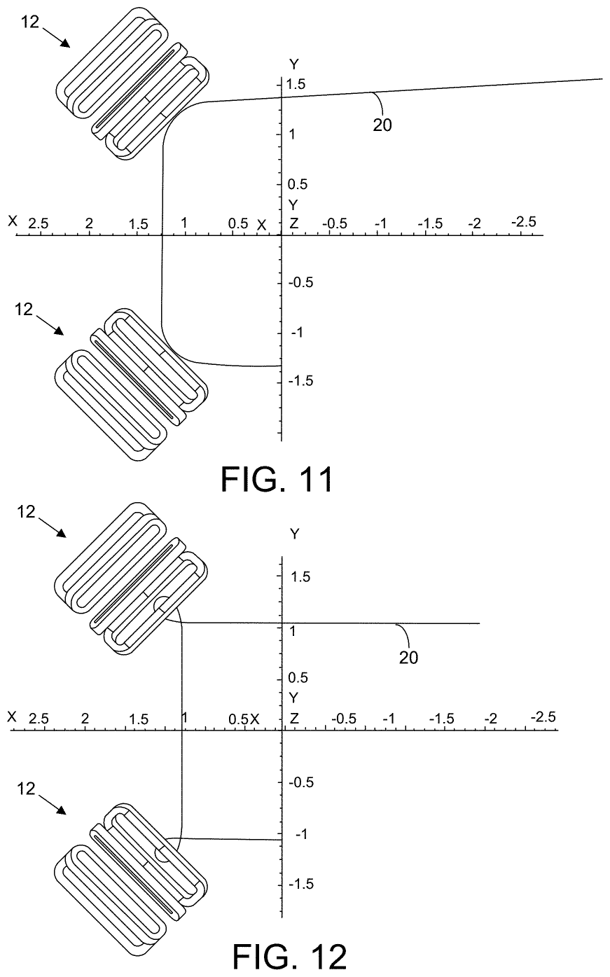

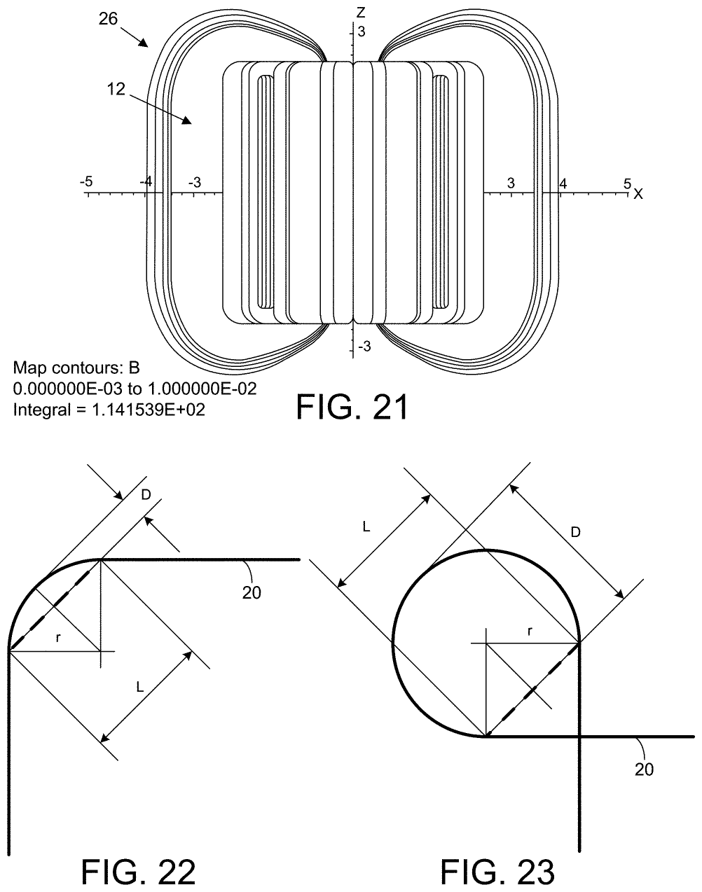

In accordance with the methods described herein, the aberrations of a toroidal bending magnet 12 are reduced by going from the traditional direct reflection of the charged beam 20 to the inverse reflection of the charged beam 20. FIGS. 11 and 12 depict trajectories of the same particles launched with the same energy into the same achromatic pair of magnets 12 differing just by the opposite field polarities reversing the direction of the current in the magnets 12. In the case of the inverse reflection [wherein the beam path penetrates (through a gap between race track coils) deeper into the magnet and loops back across the incoming path for a rotation of 270° (references to 270° in the description and claims also includes microscopic deviations therefrom—e.g., <1%—from this angle, wherein such small deviations can be fixed by focusing), as shown in FIG. 23—rather than merely being bent at a roughly 90° angle, as in direct reflection, as shown in FIG. 22], the deviation of the outgoing trajectory from the ideal path, parallel to the incoming trajectory line, is much smaller. This reduced deviation is primarily due to the fact that in the case of the inverse reflection, the particle passes the region most affected by the bowing low field area at about the same location both on the way into and out of the magnet 12, whereas, in case of the direct reflection, the particle stays in this badly shaped field area most of the time. If more than four achromatic magnets 12 are used to define the circuit, the angle of inverse reflection is accordingly adjusted (e.g, if six achromatic magnets are arranged in a hexagon, the beam 20 will be redirected by 300° via inverse reflection at each magnet).

Accordingly, using inverse reflection reduces the optical aberrations produced by the synchrotron. Extensive beam tracking modeling showed that it produces good field quality for various particles accelerated to a sufficiently wide range of energies.

### Long Coils

Another method to reduce optical aberrations (using direct reflection) is by increasing the length of the magnet 12. FIGS. 11 and 13 show the trajectories of the same particle launched at the same energy into an achromat. The length of the magnet 12 in FIG. 13 is twice the length of the magnet 12 in FIG. 11. In FIG. 13, the parallelism of the incoming and outgoing beam trajectories 20 is better, although still not perfect.

### Shaping Racetracks or Using Correction Coils

Yet another method of mitigating the bowing of the field in the beam area is by reshaping the front leg (facing the beam) of the racetrack by bending it slightly inwards, towards the magnet axis. The same effect can be achieved by using local correction coils.

Shimming with Iron

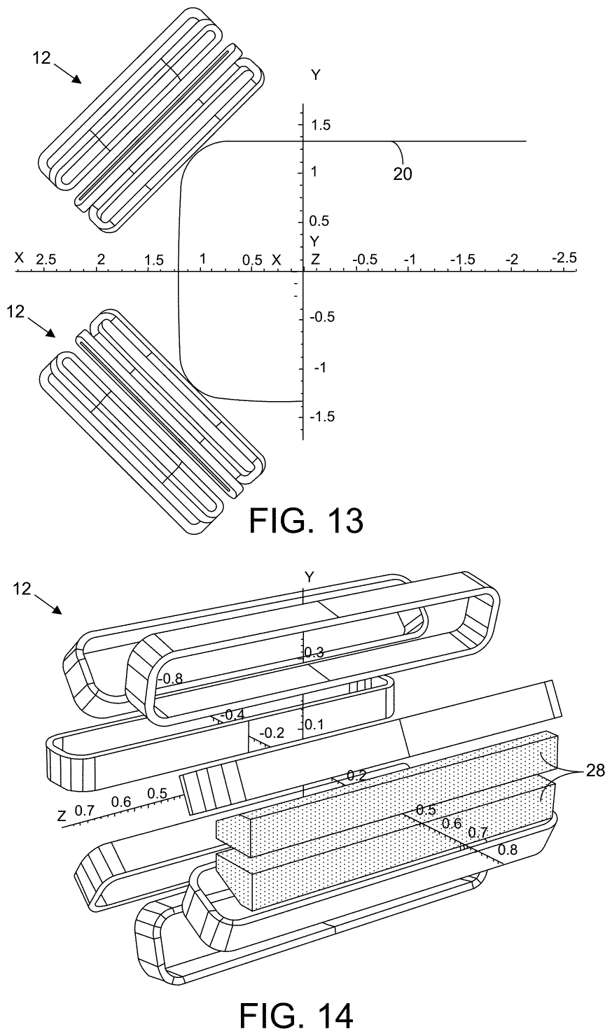

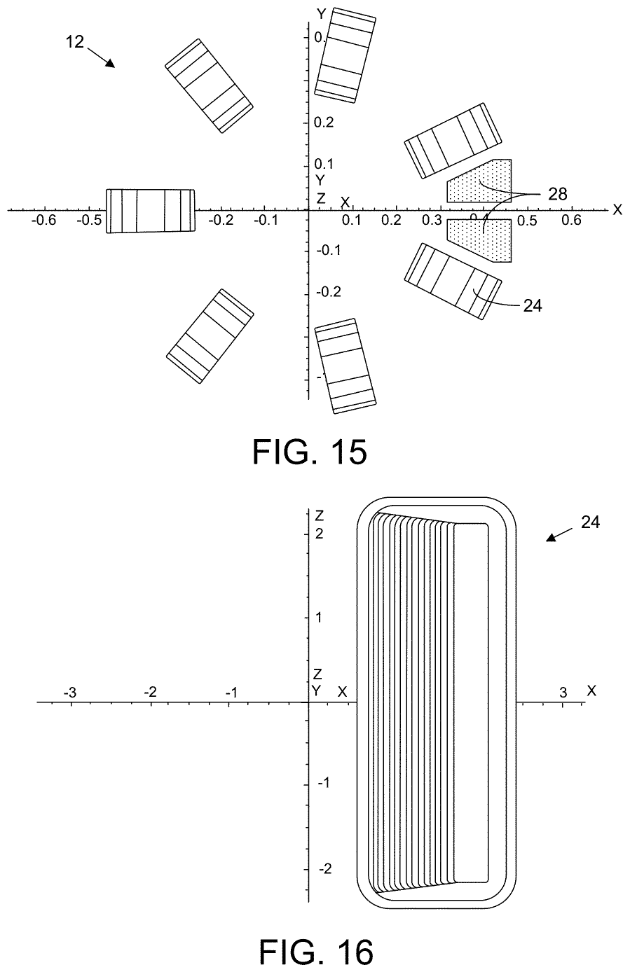

The fact that the magnet is to be maintained at the same magnetic field level opens an opportunity of shimming the space around the beam line with small amounts of magnetic or magnetizable material (e.g., iron) 28, as schematically shown in FIGS. 14 and 15.

### Constant Field Toroid

Constant field toroids (CFTs), first proposed by Dr. Mohamed A. Hilal, et al., “A prototype of a Superconductive Magnetic Energy Storage (SMES) system for Air Force use,” Air Force Development Test Center, Tyndall AFB, Fla., USA, Contract F08635-92-C-0072 (July 1992-April 1993)], were recently considered for toroidal bending magnets [L. Bromberg, P. Michael, “Toroidal Bending Magnets for Hadron Therapy Gantries,” U.S. Pat. No. 9,711,254 B2 (Jul. 18, 2017)] and for superconducting magnetic energy storage [A. Radovinsky, et al., “Constant Field Toroidal SMES Magnet,” IEEE Transactions on Applied Superconductivity, Volume: 26, Issue: 3, pp. 1-4 (April 2016); A. Radovinsky, “Inductively Decoupled Dual SMES in a Single Cryostat,” US Pub. App. No. 2015/0357104 A1 (Dec. 10, 2015)]. These publications present detailed descriptions of the features as well as design methods for designing such magnets.

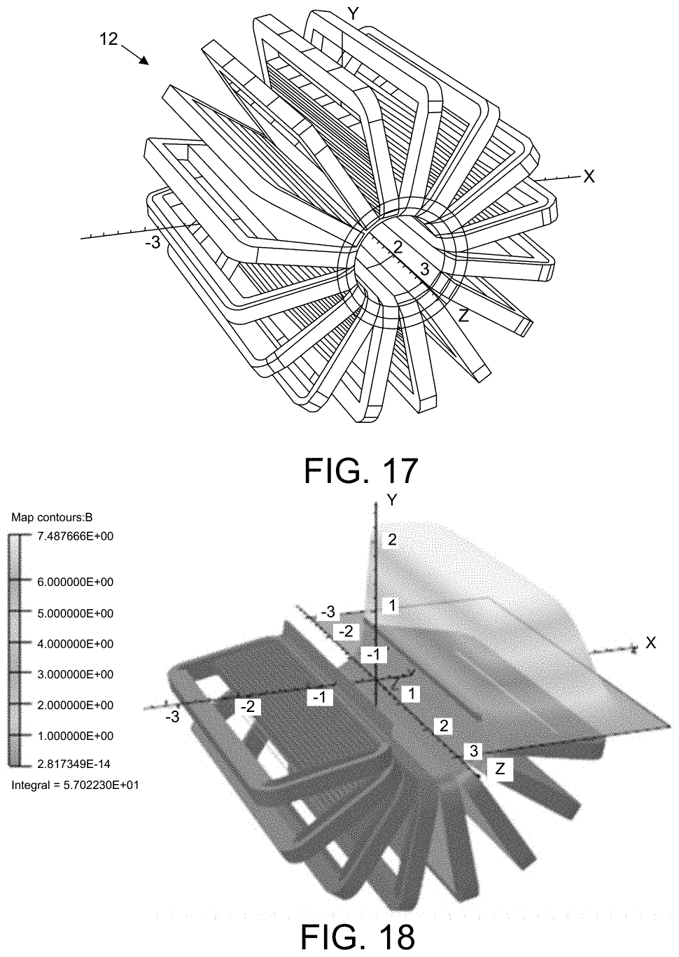

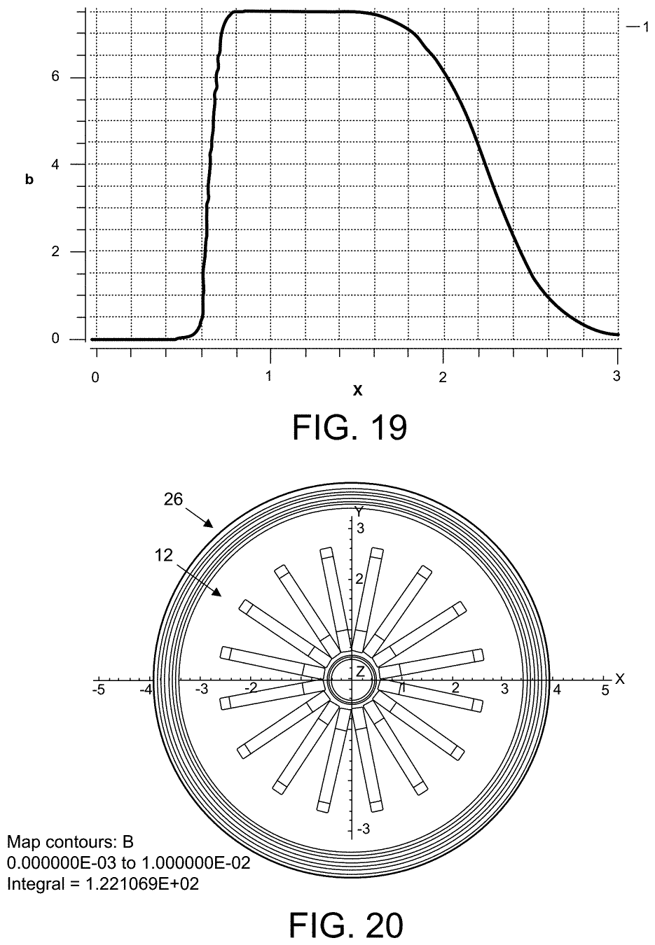

The CFT is arranged of multiple subassemblies, each comprising multiple enclosed racetracks 24. These racetracks 24 are sized and positioned in a special way, depicted in FIG. 16 so that their TF assembly, shown in FIG. 17, produces the field of the shape shown in FIGS. 18 and 19. The field in the gap between the coils is practically constant over a wide radial extent.

The field of a CFT magnet inherits the same generic properties of a traditional toroidal field (TF) magnet, including good containment of the magnetic field 26, shown in FIG. 20 and slight bowing of the field 26 along the axis, as is visible in FIG. 21.

Yet, as proven by extensive beam-tracking numerical modeling, some results of which are shown in FIGS. 3 and 4, in combination with using the inverse reflection, the magnets 12 of this type show very good beam optics for different particles accelerated to wide ranges of energies.

The penalty for the good beam optics provided by the system using the inverse reflection method is that, in this case, the depth of penetration of the particle into the toroidal field (TF) magnet 12 is significantly larger than in the case of the direct reflection. This increased penetration requires using TF magnets 12 with more bending power, accomplished either by generation of a higher magnetic field or by a larger radial build.

Generally, the build of the magnet designed to satisfy some ion- and energy-specifications is defined by beam tracking using respective computational tools. In the case of a constant-field toroid (CFT), a rough analytical estimate of the size of the working area appears to be possible.

FIGS. 22 and 23 depict the schematics of particle trajectories 18 in direct and inverse reflections, respectively. Assuming that the field changes step-wise from zero to a constant value, B, along the dashed line, we can derive the length, L, and the depth, D, of the required constant B-field beam space. The equations for doing so are as follows:

for direct reflection, L=2r/\sqrt{2}, D=r(1−1/\sqrt{2}); and

for inverse reflection, L=2r/\sqrt{2}, D=r(1+1/\sqrt{2}).

Here, r=Rₘₐₓ/B, where rigidity

$${R}_{ma x} = \frac{{Am}_{0} c}{Ze} \sqrt{{γ}_{ma x}^{2} - 1} , {γ}_{ma x} = 1 + {T}_{ma x} / {E}_{0} ,$$

where m₀=1.67262E-27 kg, e=1.60218E-19 C, E₀=938.27231 MeV. A and Z, respectively, are the atomic weight of the ion and its charge; and Tₘₐₓ is the kinetic energy of the ion at extraction.

For inverse reflection at B=6.25 T, for a 1.25 GeV proton, L=1.49 m and D=1.80 m; and for a 400 MeV carbon ion, L=1.44 m and D=1.74 m.

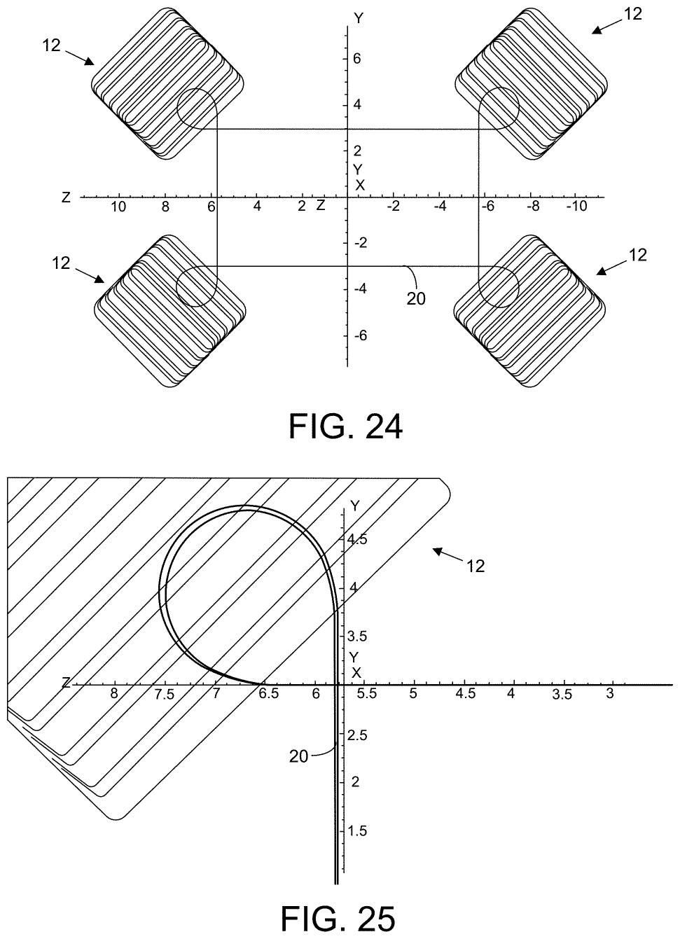

FIGS. 24 and 25 depict respective modeled beam lines 20 for the same 1.25 GeV proton and 400 MeV carbon ions in a 6.75-T CFT magnet 12, wherein the trajectory penetrating deeper into the magnet is for the protons. The agreement with the analytically estimated dimensions of the working area is not perfect because of the slope of the front edge of the field, which is clearly visible in FIGS. 18 and 19.

### Vertical Focusing

Vertical beam focusing of the bending magnets is a well-known issue. One of the most successful examples of the bending magnet with theoretically no vertical defocusing is the so-called Enge magnet [Harald A. Enge, “Achromatic Magnetic Mirror for Ion Beams,” The Review of Scientific Instruments, Vol. 34, No. 4, pp. 385-89 (1963)], i.e., a magnet with 1-D field variation, B(X)=G*Xⁿ, where G is the constant proportionality coefficient, X is the distance from the field front line, and n is the field index. This field profile, matching a “magic” n-value, provides ideal inverse reflection of a parallel beam with no vertical defocusing. In the past, the Enge magnet-field profile was known to be implemented only at low field levels by means of field shaping using ferromagnetic iron.

A physical implementation of a magnet (hereinafter, a toroidal Enge magnet or TEM) with this field profile using toroidal topology was created, and trajectories for protons in the range between 70 MeV and 230 MeV were modeled using OPERA software. The magnet demonstrated focusing properties similar to those declared by Enge. A constant-field toroid (CFT) [A. Radovinsky, et al., “Constant Field Toroidal SMES Magnet,” IEEE Transactions on Applied Superconductivity, Vol. 26, Issue 3, pp. 1-4 (April 2016)] was tested under the same conditions and showed similar but slightly worse focusing properties.

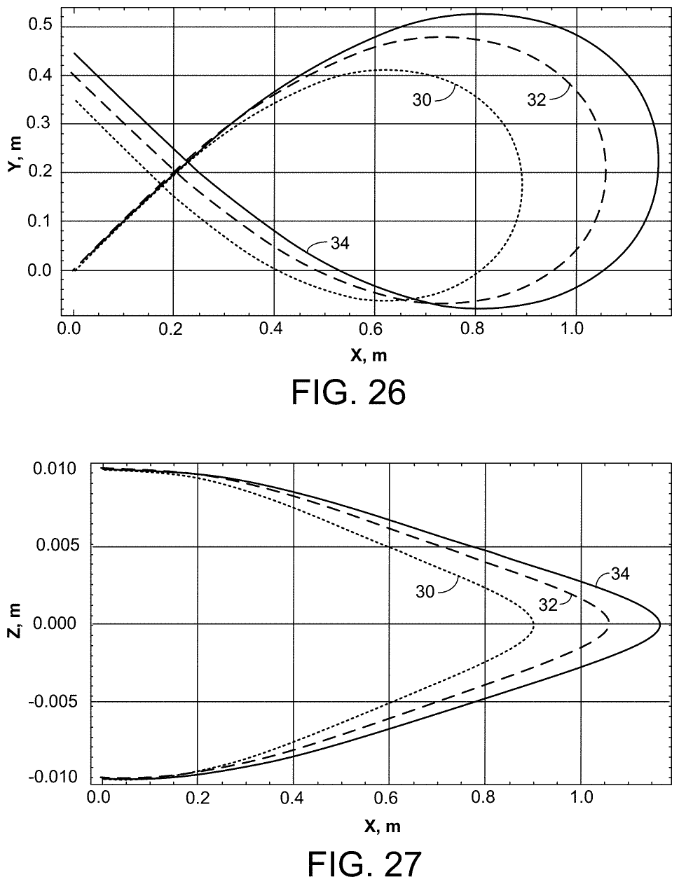

FIGS. 26 and 27 respectively depict in-plane and side-view projections of the trajectory of a proton calculated using the methods of Enge for the field profile specified by n=1.4767, G=4.8 T and protons launched at 45° from the (X, Y, Z)=(0,0,1 cm) with energies, 70 MeV (plot 30), 150 MeV (plot 32), and 230 MeV (plot 34).

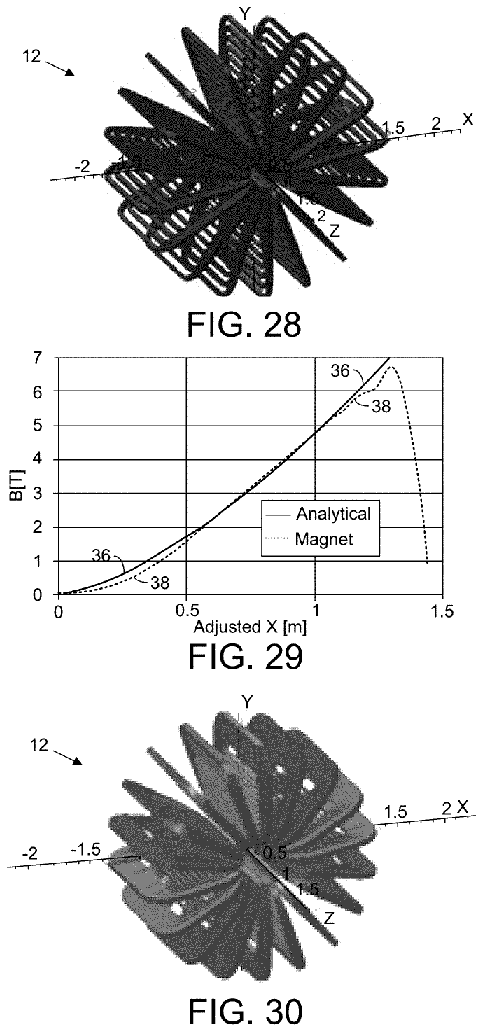

A toroidal Enge magnet 12 matching the same definitions, B(X)=G*Xⁿ (n=1.4767, G=4.8 T), was created and is shown in FIG. 28. FIG. 29 shows the analytical profile 36 and in the as-modeled field profile 38. For the latter, the X=0 point is adjusted; it is placed at X=−1.6 m in the coordinates of the physical magnet.

Note that in the interval, 0<X<0.5 m, matching is not perfect. The inevitability of this deviation in practical implementations was admitted by Enge.

Besides the toroidal Enge magnet (TEM), a constant field toroid (CFT) 12 was also created (shown in FIG. 30), and the beam focusing properties of these two magnets were compared for the same conditions.

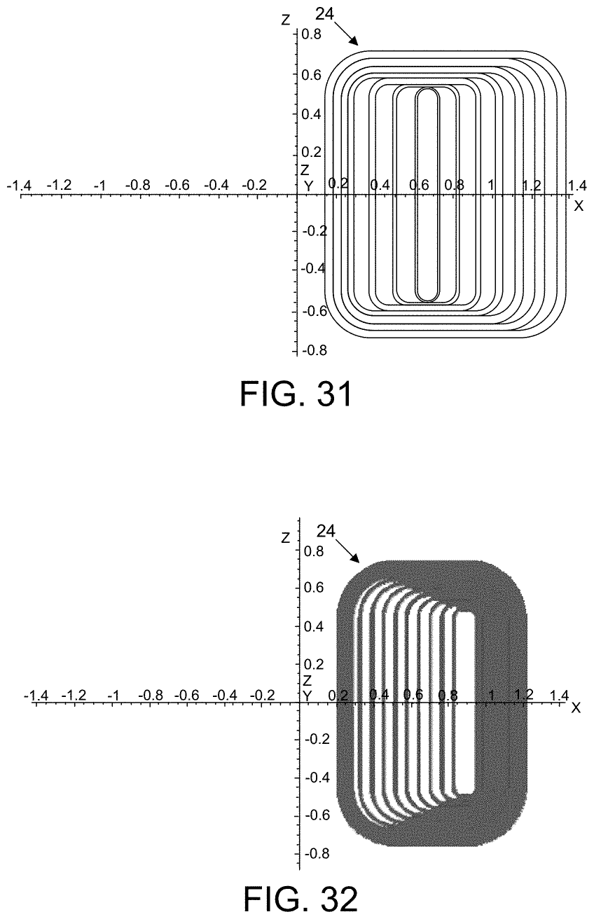

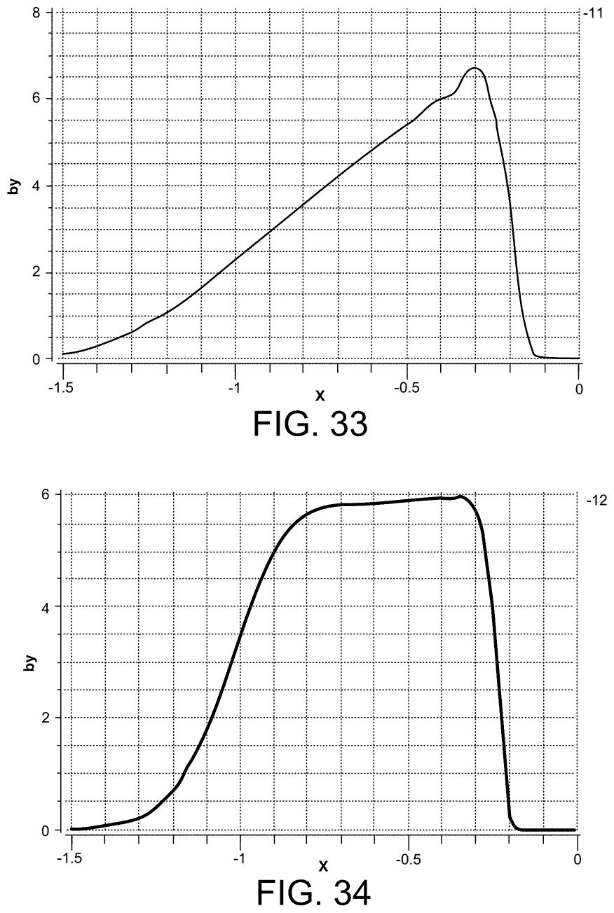

Both magnets 12 shown in FIGS. 28 and 30 have the same rotational symmetry. Each comprises 18 sub-coils 24 formed by racetracks, as shown in respective FIGS. 31 and 32. FIGS. 33 and 34 show field profiles along respective (−1.5 m<X<0, Y=Z=0) lines. The coordinate systems are centered at the centers of the magnets.

Peak fields on the conductors are about 7.7 T in both cases. In both magnets 12, smeared current density is 80 A/mm². Total volume of the conductor in the magnet is 0.75 m³ and 1.04 m³ for the TEM and the CFT, respectively. Stored EM energy is 29.7 MJ and 42.8 MJ for the TEM and the CFT, respectively.

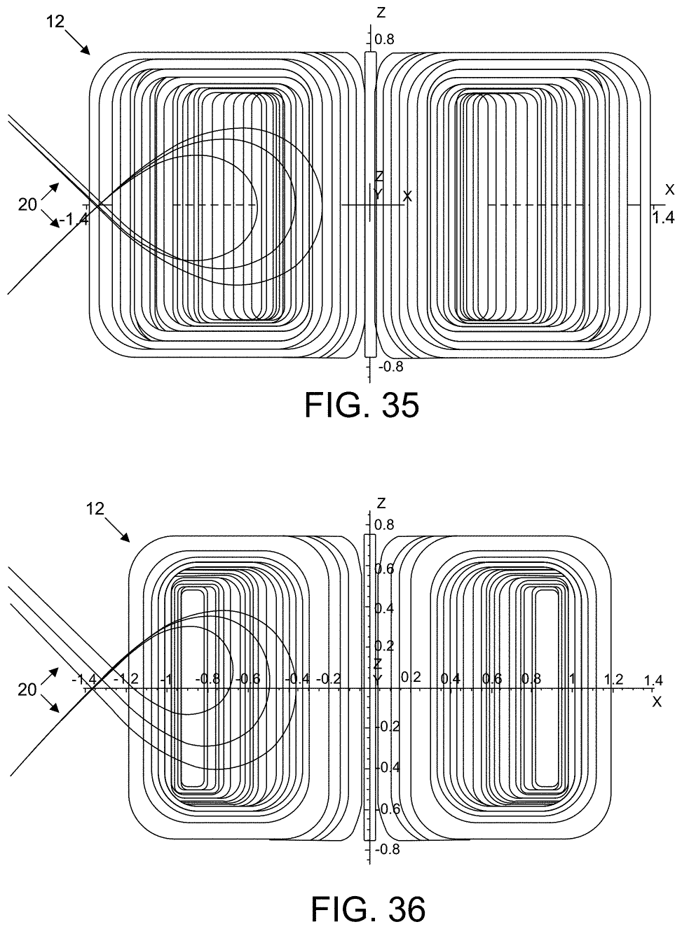

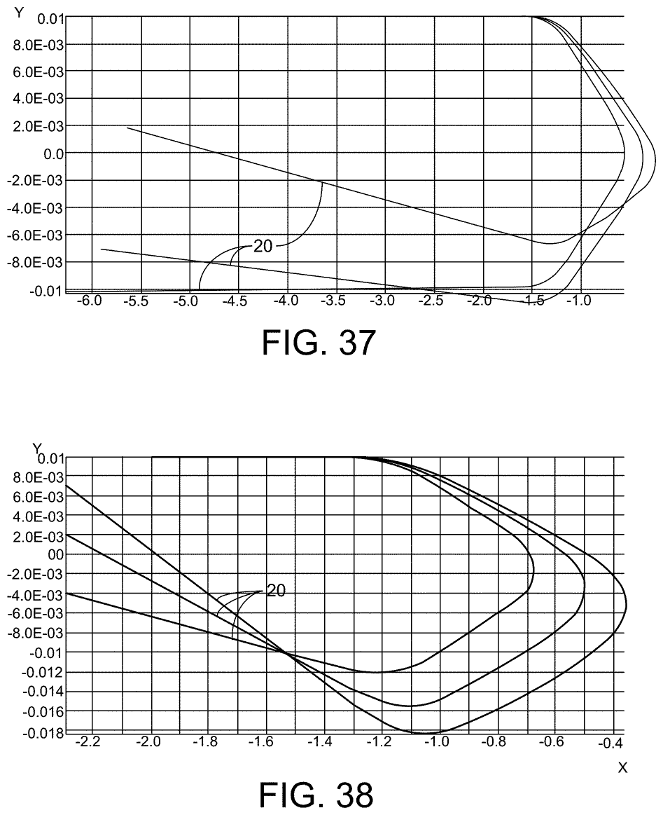

FIGS. 35-38 depict in-plane, Z(X), and out-of-plane, Y(X), projections of the trajectories 20 of protons launched parallel to the XZplane at 45° from the point with coordinates (X, Y, Z)=(−2 m, 1 cm, −0.65 m) with respective energies, 70 MeV, 150 MeV and 230 MeV. Protons with higher energy penetrate deeper in X-direction.

The focusing properties of both magnets are comparable. In both cases, reflected particles return within the same |Y|<1 cm range, showing very small vertical defocusing up to substantial distance from the magnet. In the TEM, the beam is focused at a longer distance from the magnet.

Also, the TEM has a stored energy that is smaller than that of the CFT and the volume of the superconducting winding smaller than the volume of the superconductor of the CFT winding. On the other hand, the overall dimensions of the CFT (diameter, D=2.4 m, and length, L=1.5 m) are smaller than those of the TEM (D=2.8 m, L=1.5 m).

### RF Drive

Let us assume that the particles are accelerated by a pair of acceleration cavities (gaps) installed at some points along the long sides of the rectangle formed by the beam trajectory. Let us assume that the voltage across the cavity is V; and we assume, in each cavity, that the voltage varies harmonically with the frequency, f(t). Passing through the cavity, a charged particle, characterized by A and Z≠0, gains additional kinetic energy per nucleon, dT=V(Z/A). Let us assume that the length, L, of the trajectory between the cavities is about the same (i.e., 2L for the whole revolution). The kinetic energy of the particle between the cavities stays the same so that, after the nth cavity, it amounts to Tₙ=nV(Z/A). This corresponds to the velocity, ϑₙ=c\sqrt{1−1/γₙ²}, where γₙ=1+Tₙ/E₀. The time spent by the particle to pass the distance between the cavities is τₙ=L/ϑ₂. Time of flight accumulates as t=Σₙτₙ. The harmonic frequency of the voltage in each of two cavities is, fₙ=1/(2τₙ). It varies as a function of the time of flight, t.

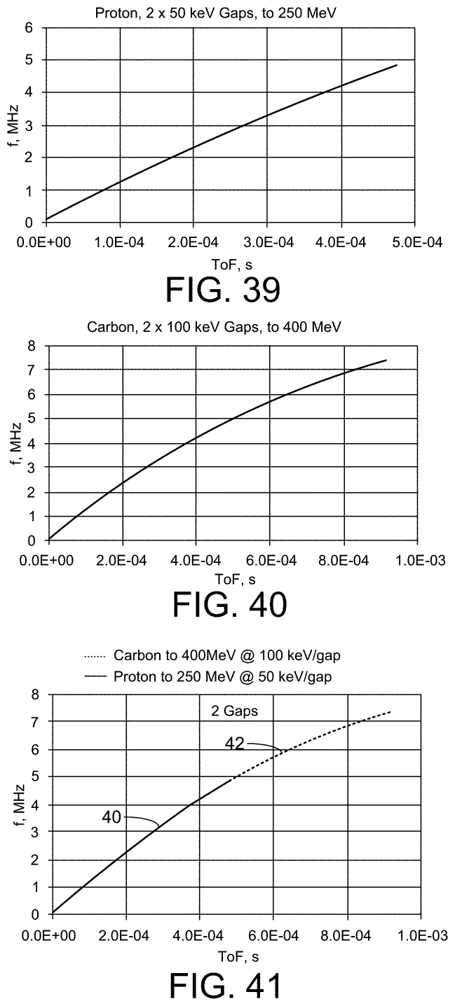

FIGS. 39 and 40 show the RF frequency, f(ToF), versus time of flight, ToF, for a proton and a carbon ion accelerated in the same device with L=14.5 m to 250 MeV and 400 MeV respectively using respective 50 keV and 100 keV voltages in each of two cavities.

The overlay of proton 40 and carbon ion 42 curves, shown in FIG. 41, indicates that, given the gap voltage is scaled by the (Z/A) multiplier, the frequency versus time functions overlay exactly. This relationship implies that the same frequency drive, tuned for the longest cycle, can be used for accelerating different particles as long as the gap voltages are scaled by (Z/A) and for scenarios with a shorter cycle during the part of the cycle the drive is deactivated by nullifying the gap voltage.

### Injection and Extraction

Methods of beam injection and extraction can also be inherited from the traditional methods used for the design of synchrotrons.

Beam tracking showed that the acceptance of the magnetic system is good within a wide range of energies, starting from 50 keV for protons and from 100 keV for the carbon ions. These minimum values were chosen under assumption that, for the carbon, the acceleration voltage per cavity can reach 100 keV. A compatible RF system used for accelerating protons will operate at 50 keV per cavity.

Another option for implementing beam injection is by using preaccelerators, such as linear accelerators (linacs) or small cyclotrons.

More modeling, not shown here, indicated that systems with direct reflection can provide good optics only in a limited range of beam energies. These systems can be tuned for low energy but will start producing aberrations growing with the particle energy. That is why using direct reflection may be viewed as being inferior to inverse reflection.

Ion extraction from the cavity can be achieved either by a kicker magnet or by an electrostatic deflector. The latter method appears to be more promising.

### Implications for Toroidal Bending Magnets

As indicated, above, using toroidal magnets, in general, and CFT magnets, in particular, as bending magnets in hadron therapy gantries, was proposed in U.S. Pat. No. 9,711,254 B2 (Bromberg, et al.), where the possibility of arranging them in an achromatic configuration was presented as an advantage of that invention. U.S. Pat. No. 9,711,254 B2, however, considered only the direct reflection scheme without mentioning the possibility of using inverse reflection. Results of the modeling related to the present study indicated that achromatic systems using direct reflection have difficulty producing the same high-quality optics through wide ranges of beam energies, particularly at low energies.

Using inverse reflection for bending magnets and their achromatic arrangements can significantly improve their optics and reduce aberrations related to the variation of the beam energy. The advantages of the inverse-reflection ion acceleration are applicable to the achromatic bending magnets used in the whole range of angles, from very small to 180-degrees, as described herein.

### Implications for Hadron Radiotherapy Treatment Systems

Two main magnetic subsystems of a hadron radiotherapy treatment (HRT) system are the accelerator and the gantry delivering the beam from the accelerator to the patient. Using achromatic dipoles comprising toroidal bending magnets was proposed in U.S. Pat. No. 9,711,254 B2. Usually, the energy of the charged particles delivered to the patient is in the range excluding small energies. For protons, the energy of the charged particles is from about 70 MeV to 250 MeV.

Traditional achromatic bending magnets provide good optics in a rather narrow energy range. For wider beam-energy ranges, the field in the achromat is changed, which takes some time and extends the time of a fractional treatment. The acceptance of an achromatic TBM can produce sufficiently good optics within a wide energy range, so that it can be used in a DC mode without varying its field.

This capability creates an opportunity of combining the proposed DC accelerator with the gantry using DC achromatic toroidal bending magnets and subsequently removes any need to sweep the field in any significant magnets of the HRT system, resulting in much-faster beam-energy variation and in-depth beam scanning, limited only by the electronics.

### Miscellaneous

Herein, achromats using superconducting toroidal bending magnets (TBMs) were particularly discussed for use in the proposed synchrotrons. In fact, achromatic magnets of any type, superconducting or normal, can be used for this purpose, as long as they are capable of producing the same functionality as TBMs (i.e., they have the same acceptance within the whole range of beam energies through the whole acceleration cycle). Alternatively, if the acceptance of such achromats at low energies is insufficient, an additional external accelerator can be used to pre-accelerate the charged particles to the energy acceptable for the proposed system.

In describing embodiments of the invention, specific terminology is used for the sake of clarity. For the purpose of description, specific terms are intended to at least include technical and functional equivalents that operate in a similar manner to accomplish a similar result. Additionally, in some instances where a particular embodiment of the invention includes a plurality of system elements or method steps, those elements or steps may be replaced with a single element or step. Likewise, a single element or step may be replaced with a plurality of elements or steps that serve the same purpose. Further, where parameters for various properties or other values are specified herein for embodiments of the invention, those parameters or values can be adjusted up or down by 1/100^{th}, 1/50^{th}, 1/20^{th}, 1/10^{th}, ⅕^{th}, ⅓^{rd}, ½, ⅔^{rd}, ¾^{th}, ⅘^{th}, 9/10^{th}, 19/20^{th}, 49/50^{th}, 99/100^{th}, etc. (or up by a factor of 1, 2, 3, 4, 5, 6, 8, 10, 20, 50, 100, etc.), or by rounded-off approximations thereof, unless otherwise specified. Moreover, while this invention has been shown and described with references to particular embodiments thereof, those skilled in the art will understand that various substitutions and alterations in form and details may be made therein without departing from the scope of the invention. Further still, other aspects, functions, and advantages are also within the scope of the invention; and all embodiments of the invention need not necessarily achieve all of the advantages or possess all of the characteristics described above. Additionally, steps, elements and features discussed herein in connection with one embodiment can likewise be used in conjunction with other embodiments. The contents of references, including reference texts, journal articles, patents, patent applications, etc., cited throughout the text are hereby incorporated by reference in their entirety for all purposes; and all appropriate combinations of embodiments, features, characterizations, and methods from these references and the present disclosure may be included in embodiments of this invention. Still further, the components and steps identified in the Background section are integral to this disclosure and can be used in conjunction with or substituted for components and steps described elsewhere in the disclosure within the scope of the invention. In method claims (or where methods are elsewhere recited), where stages are recited in a particular order—with or without sequenced prefacing characters added for ease of reference—the stages are not to be interpreted as being temporally limited to the order in which they are recited unless otherwise specified or implied by the terms and phrasing.

What is claimed is:

1. A method for accelerating charged particles, comprising:
in a direct-current synchrotron that comprises a plurality of achromatic magnets arranged in a continuous sequence and defining an acceleration device, directing a beam of charged particles toward one of the achromatic magnets;
allowing the charged-particle beam to penetrate a gap in the achromatic magnet;
repeatedly redirecting the charged-particle beam through an arc of at least 270° via inverse reflection at each of the achromatic magnets to produce a series of beam lines that form a circuit in which the charged-particle beam is accelerated over successive passes through the circuit;
generating a substantially constant-in-time magnetic field with the achromatic magnets over the repeated redirections of the charged-particle beam; and
extracting the charged particles from the acceleration device.

2. The method of claim 1, wherein the achromatic magnets are toroidal magnets.

3. The method of claim 2, wherein the toroidal magnets comprise a superconductor.

4. The method of claim 3, wherein the superconductor is a high-temperature superconductor.

5. The method of claim 2, wherein the toroidal magnets are electrically coupled with a DC power source that delivers constant direct current through the superconductor in the toroidal magnets.

6. The method of claim 2, wherein the toroidal magnets are oriented at an angle relative to the beam lines, wherein that angle is the quotient of 180° divided by the number of achromatic magnets.

7. The method of claim 2, wherein the toroidal magnets comprise a plurality of race track coils, and wherein the charged-particle beam passes between a pair of the race track coils as the charged-particle beam is redirected.

8. The method of claim 7, wherein the toroidal magnets further comprise magnetic or magnetizable shims on opposite sides of the circuit between race track coils.

9. The method of claim 1, further comprising focusing the charged-particle beam with focusing multipole magnets as the charged-particle beam passes through the circuit.

10. The method of claim 1, further comprising directing different charged particles through the circuit without retuning the achromatic magnets.

11. The method of claim 10, further comprising only adjusting a voltage applied to an acceleration cavity in the circuit when the charged particles are changed.

12. The method of claim 1, wherein the circuit comprises two sets of collinear parallel beam lines.

13. The method of claim 1, further comprising using the charged particles for performing hadron therapy on a human patient after extraction.

14. A direct-current synchrotron, comprising:
a plurality of achromatic magnets configured to define a circuit for charged-particle acceleration, wherein the achromatic magnets are configured to generate inverse reflection; and
an acceleration cavity configured to accelerate charged particles in the circuit.

15. A method for bending a path for charged particles, comprising:
directing a charged-particle beam along a first beam line toward a magnet generating a constant-in-time magnetic field; and
redirecting the path of the charged-particle beam to produce a second beam line with the magnet via inverse reflection.

16. The method of claim 15, wherein charged particles are of different energy, charge and mass.

17. The method of claim 15, wherein the magnet is a constant-field toroidal magnet comprising a superconductor.

18. The method of claim 15, wherein the magnet is a toroidal magnet comprising a superconductor and generates an Enge field profile.

19. The method of claim 15, further comprising bending the path further with a second magnet to produce a third beam line via inverse reflection, wherein the first and third beam lines are collinear at different energies of the particles of the same charge and mass.

20. The method of claim 19, further comprising directing different ions with different energies along the first beam line, the second beam line, and the third beam line with the magnets.
